(19) United States
(12) Patent Application Publication
(10) Pub. No.: US 2006/0032496 A1
(43) Pub. Date: Feb. 16, 2006

(54) Inhalation actuated percussive ignition system

(75) Inventors: Ron L. Hale, Woodside, CA (US); Peter M. Lloyd, Walnut Creek, CA (US)

Correspondence Address: SWANSON & BRATSCHUN, L.L.C, 1745 SHEA CENTER DRIVE, SUITE 330, HIGHLANDS RANCH, CO, 80129, US

(73) Assignee: Alexza Molecular Delivery Corporation, Palo Alto, CA (US)

(21) Appl. No.: 10/917,735

(22) Filed: Aug. 12, 2004

(51) Int. Cl. A61M 11/00

(52) U.S. Cl. 128/200.23; 128/200.14

(57) ABSTRACT

Percussive ignition systems and heat packages incorporating percussively igniter systems capable of being activated by inhalation are disclosed.

This disclosure relates to percussive ignition systems capable of being actuated by inhalation, percussively activated heating elements, and the use of inhalation actuated percussive ignition systems for activating heating elements.

Pulmonary delivery is known as an effective way to administer physiologically active compounds to a patient for the treatment of diseases and disorders. Devices developed for pulmonary delivery generate an aerosol of a physiologically active compound that can be inhaled by a patient where the compound can be used to treat conditions in a patient's respiratory tract and/or enter the patient's systemic circulation. Devices for generating aerosols of physiologically active compounds include nebulizers, pressurized metered-dose inhalers, and the dry powder inhalers. Nebulizers are based on atomization of liquid drug solutions, while pressurized metered-dose inhalers and dry powder inhalers are based on suspension and dispersion of dry powder in an airflow.

Aerosols for inhalation of physiologically active compounds can also be formed by vaporizing a substance to produce a condensation aerosol comprising the active compounds in an airflow. A condensation aerosol is formed when a gas phase substance condenses or reacts to form particulates. Examples of devices and methods employing vaporization methods to produce condensation aerosols are disclosed in U.S. application Ser. No. 10/861,554, entitled “Multiple Dose Condensation Aerosol Devices and Methods of Forming Condensation Aerosols, filed Jun. 3, 2004, and U.S. Application Ser. No. 10/850,895, entitled “Self-Contained Heating Unit and Drug-Supply Unit Employing Same,” filed May 20, 2004, each of which is incorporated herein by reference.

Efficient production of a condensation aerosol comprising a drug is facilitated by rapidly vaporizing the drug such that there is minimal degradation of the drug. The vaporized drug can condense to produce an aerosol characterized by high yield and purity. For use in medical devices, it is useful that the heat source for vaporizing the drug be compact and capable of producing a rapid heat impulse. Chemically based heating units can include a fuel which is capable of undergoing an exothermic metal oxidation-reduction reaction within an enclosure, such as those described in, for example, U.S. application Ser. No. 10/850,895 entitled “Self-Contained Heating Unit and Drug-Supply Unit Employing Same,” filed May 20, 2004, the entirety of which is herein incorporated by reference.

A fuel can be ignited to generate a self-sustaining oxidation-reduction reaction. Once a portion of the fuel is ignited, the heat generated by the oxidation-reduction reaction can ignite adjacent unburned fuel until all of the fuel is consumed in the process of the chemical reaction. The exothermic oxidation-reduction reaction can be initiated by the application of energy to at least a portion of the fuel. Energy absorbed by the fuel or by an element in contact with the solid fuel can be converted to heat. When the fuel becomes heated to a temperature above the auto-ignition temperature of the reactants, e.g., the minimum temperature required to initiate or cause self-sustaining combustion in the absence of a combustion source or flame, the oxidation-reduction reaction will initiate, igniting the solid fuel in a self-sustaining reaction until the fuel is consumed.

The auto-ignition temperature of a solid fuel comprising a metal reducing agent and a metal-containing oxidizing agent as disclosed in U.S. application Ser. No. 10/850,895 entitled “Self-Contained Heating Unit and Drug-Supply Unit Employing Same,” can range from 400° C. to 500° C. While such high auto-ignition temperatures facilitate safe processing and safe use of the fuel under many use conditions, for example, as a portable medical device, for the same reasons, to achieve such high temperatures, a large amount of energy must be applied to the fuel to initiate the self-sustaining reaction.

As is well known in the art, for example, in the pyrotechnic industry, sparks can be used to safely and efficiently ignite fuel compositions. Sparks refer to an electrical breakdown of a dielectric medium or the ejection of burning particles. In the first sense, an electrical breakdown can be produced, for example, between separated electrodes to which a voltage is applied. Sparks can also be produced by ionizing a compound in an intense electromagnetic field. Examples, of burning particles include those produced by friction and break sparks produced by intermittent electrical current. Sparks of sufficient energy incident on a fuel can initiate the self-sustaining oxidation-reduction reaction.

Compact initiator compositions and igniters using electrically resistive heating to ignite the sparking compositions capable of igniting metal oxidation/reduction fuels, which produce low amounts of gas as appropriate for enclosed systems, and which do not contain explosive material as classified by the Department of Transportation for use in medical, food, and other such devices are described, for example, in U.S. application Ser. No. 10/851,018 entitled “Stable Initiator Compositions and Igniters,” the entirety of which is incorporated herein by reference. Batteries are used to provide power to the electrically resistive heaters used in such devices. Batteries can be expensive, bulky, and also create disposal issues.

Percussive mechanisms can also be used to ignite initiator compositions. For example, percussive ignition systems are used in the photographic industry, as described, for example, in U.S. Pat. No. 3,724,991. A photoflash lamp includes a sealed light-transmitting envelope containing a combustion-supporting gas such as oxygen together with a light producing combustible material such as zirconium, aluminum, or hafnium. In a percussively ignited photoflash lamp, a charge of percussively sensitive initiator material is located within a readily deformable metal ignition tube, sealed within and projecting from one end of a length of glass tubing which forms the envelope containing the fuel. The initiator composition can be coated on a wire anvil supported within the ignition tube, or can be deposited within the deformable tube. The initiator composition is ignited by a mechanical impact to the tube sufficient to deform the tube. The compressive force on the initiator composition causes deflagration of the initiator composition. Sparks generated by the burning initiator composition are propelled through the tube to ignite the fuel in the envelope.

Over the years of use in the photographic industry, percussive ignition systems are shown to be small, safe, reliable, and amenable to high volume manufacturing. Percussive ignition systems for use in portable medical devices and in particular, aerosol inhalation medical devices have been disclosed in U.S. application Ser. No. 10/851,883, entitled “Percussively Ignited or Electrically Ignited Self-Contained Heating Unit and Drug Supply Unit Employing Same,” filed May 20, 2004, the entirety which is incorporated herein by reference. However, such systems using inhalation actuation to mechanically impact the igniter and/or containment of the igniter anvil and fuel in a single enclosure have not been previously described. With the advent of portable medical devices capable of providing high purity drug aerosols upon rapid vaporization of a thin film of drug, wherein a metal/oxidation reduction reaction provides a high temperature thermal impulse, there is a need for percussive ignition systems that can be actuated by inhalation.

Certain aspects of the present disclosure provide inhalation actuated percussive ignition systems comprising, a housing defining an airway, wherein the housing comprises at least one air inlet and a mouthpiece having at least one air outlet, an airflow sensitive actuator coupled to the airway, and a mechanism coupled to the airflow sensitive actuator configured to activate a percussive igniter, wherein the percussive igniter is activated by an air flow in the airway produced by inhaling through the mouthpiece.

A second aspect of the present disclosure provides methods for activating a percussive igniter, comprising the steps of, providing an inhalation actuated percussive ignition system, inhaling through a mouthpiece to generate an air flow in an airway, actuating an airflow sensitive actuator, and activating a percussive igniter.

A third aspect of the present disclosure provides a percussively activated heating element by inhalation comprising an enclosure comprising a region capable of being deformed by a mechanical impact, an anvil disposed within the enclosure, and a percussive initiator composition disposed within the enclosure, wherein the initiator composition is configured to be ignited when the deformable region of the enclosure is deformed, and a fuel disposed within the enclosure configured to be ignited by the initiator composition.

A fourth aspect of the present disclosure provides inhalation actuated heating systems comprising a housing defining an airway, wherein the housing comprises at least one air inlet and a mouthpiece having at least one air outlet, an airflow sensitive actuator coupled to the airway, a mechanism coupled to the airflow sensitive actuator configured to activate a percussive igniter, and a heating element comprising a fuel, wherein the fuel is configured to be ignited by the percussive igniter, wherein the percussive igniter is activated by an air flow in the airway produced by inhaling through the mouthpiece.

A fifth aspect of the present disclosure provides methods for actuating a percussively activated heat package comprising inhaling to generate an airflow, actuating an airflow sensitive actuator coupled to the air flow, activating a percussive igniter coupled to the air flow sensitive actuator, and igniting a fuel produce heat.

A sixth aspect of the present disclosure provides methods for producing a condensation aerosol of a substance using an inhalation actuated percussively activated heating element.

It is to be understood that both the foregoing general description and the following detailed description are exemplary and explanatory only and are not restrictive of certain embodiments, as claimed.

### DESCRIPTION OF THE DRAWINGS

FIG. 1 is an illustration of a percussive igniter.

FIGS. 2A-2D are illustrations of an inhalation actuated percussive ignition system according to certain embodiments.

FIGS. 3A-3B are illustrations of an actuation mechanism comprising a diaphragm for activating a percussive igniter according to certain embodiments.

FIGS. 4A-4D are illustrations of percussively activated heat packages according to certain embodiments.

FIG. 5 is an illustration of another embodiment of a heat package.

FIG. 6 is an illustration of still another embodiment of a heat package.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### DESCRIPTION OF VARIOUS EMBODIMENTS

Unless otherwise indicated, all numbers expressing quantities and conditions, and so forth used in the specification and claims are to be understood as being modified in all instances by the term “about.”

In this application, the use of the singular includes the plural unless specifically stated otherwise. In this application, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

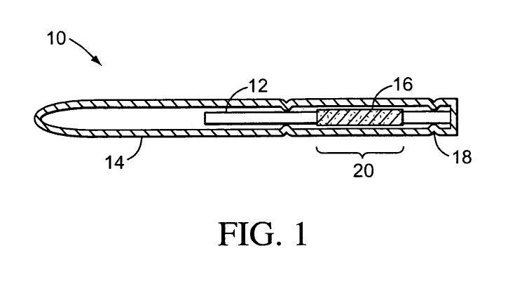

A percussive igniter comprises a deformable part, an anvil disposed adjacent the deformable part, and an initiator composition disposed between the anvil and the deformable part. In some embodiments an anvil within the percussive igniter is not required. The initiator composition is activated by a mechanical impact or force sufficient to compress the initiator composition between the deformable part and the anvil. An example of a percussive igniter is illustrated in FIG. 1. FIG. 1 shows a percussive igniter 10 having an anvil 12, coaxially disposed within a deformable enclosure 14. A portion of the exterior of anvil 12 is coated with an initiator composition 16. Anvil 12 is held in place by indentations 18 which maintain initiator composition 16 adjacent to but not in contact with the inner surface of enclosure 14. A sufficient mechanical impact or force applied to the outside wall of enclosure 14 in the region adjacent initiator composition 16, identified as region 20, can cause region 20 to deform, compressing initiator composition 16 against anvil 12. The compressive force can ignite initiator composition 16 to deflagrate and eject sparks. The sparks can in turn be used to ignite a fuel (not shown). In certain embodiments, the initiator composition can be directly coated or placed inside the deformable enclosure as opposed to use of a coated anvil.

In certain embodiments, enclosure 14 can comprise a metal tube that can deform upon application of an impact force ranging from about 0.5 in-lb to about 3.0 in-lb. As will be appreciated by one of skill in the art the amount of impact force to be applied will be limited by the strength of the tube and the holder and can be readily determined. The thickness and material forming the tube can be such that the tube reliably deforms upon impact within a specific range of force, but will not distort under normal use conditions. In certain embodiments, the tube can be formed from a metal such as aluminum, nickel-chromium iron alloy, brass or steel and can have a wall thickness ranging from about 0.001 inches to about 0.005 inches. In certain embodiments, the tube can also maintain structural integrity when impacted such that the walls will not perforate or tear when deformed. In certain embodiments, the enclosure can comprise a stainless steel tube having a thickness of 0.005 inches±0.001 inches, and a diameter of about 0.58 inches that is capable of deforming upon impact with a force of at least 0.75 in-lb. Other materials, dimensions, and shapes for the enclosure can also be used and/or optimized for specific applications.

In certain embodiments, anvil 12 can comprise a non-compressible rod, pin, or wire. Anvil 12 can be solid material that can provide a surface upon which initiator composition 16 can be compressed when the deformable part is impacted. The material forming anvil 12 can be, for example, a metal, alloy, ceramic, plastic, composite or the like. The diameter of anvil 12 will be slightly smaller than the inner diameter of deformable tube 14. For example, anvil 12 can have a diameter about 0.01 inches less than the inner diameter of the tube. At least a part of anvil 12 is provided with a coating of a percussively activated initiator composition 16. The thickness of the coating of initiator composition 16 can range from about 0.001 inches to about 0.05 inches. The thickness of the coating can be any appropriate thickness to provide sparks for an intended application. Anvil 12 can be position within tube 14 such that the surface exterior surface of initiator composition 16 is separated from the inner wall of tube 14 by a few thousandths of an inch, for example, about 0.004 inches. Anvil 12 can be positioned within tube 14 to provide a clearance of a few thousandths of an inch between initiator composition 16 and the inner wall of tube 14. In certain embodiments, anvil 12 can be coaxially disposed within tube 14. Other thicknesses of the initiator composition and dimensions of the anvil with respect to the inner dimensions of an enclosure can be determined and/or optimized for specific applications and use conditions.

Anvil 12 can be positioned within deformable enclosure 14 and supported such that clearance is maintained between the coating of initiator composition 16 and the inner wall of the enclosure. The position of anvil 12 can be maintained, for example, by crimps, indentations, protuberances, gaskets, inserts, and the like. Devices for positioning anvil 12 can be separate, or can be integral to anvil 12. In FIG. 1, indentations 18 hold anvil 12 coaxially within enclosure 14. In certain embodiments, it can be desirable that any anvil positioning element be non-combustible, maintain integrity at high temperatures, and in certain embodiments, be thermally non-conductive. Crimps, indentations or protuberances used to maintain the position of anvil 12 can extend the circumference of anvil 12, or be discrete such that one or more spaces or gaps is provided between anvil 12 and enclosure 14. The spaces or gaps can provide an essentially unobstructed region through which sparks generated by deflagration of initiator composition 16 can be propelled. The anvil positioning features can contact anvil 12 in a region of anvil 12 not coated with initiator composition 16.

Percussively activated initiator compositions are well known in the art. Initiator compositions for use in a percussive ignition system will deflagrate when impacted to produce intense sparking that can readily and reliably ignite a fuel such as a metal oxidation-reduction fuel. For use in enclosed systems, such as for example, for use in heat packages, it can be useful that the initiator compositions not ignite explosively, and not produce excessive amounts of gas. Certain initiator compositions are disclosed in U.S. patent application Ser. No. 10/851,018, entitled “Stable Initiator Compositions and Igniters,” the entirety of which is incorporated herein by reference. Initiator compositions comprise at least one metal reducing agent, at least one oxidizing agent, and optionally at least one inert binder.

In certain embodiments, a metal reducing agent can include, but is not limited to molybdenum, magnesium, phosphorous, calcium, strontium, barium, boron, titanium, zirconium, vanadium, niobium, tantalum, chromium, tungsten, manganese, iron, cobalt, nickel, copper, zinc, cadmium, tin, antimony, bismuth, aluminum, and silicon. In certain embodiments, a metal reducing agent can include aluminum, zirconium, and titanium. In certain embodiments, a metal reducing agent can comprise more than one metal reducing agent.

In certain embodiments, an oxidizing agent can comprise oxygen, an oxygen based gas, and/or a solid oxidizing agent. In certain embodiments, an oxidizing agent can comprise a metal-containing oxidizing agent. Examples of metal-containing oxidizing agents include, but are not limited to, perchlorates and transition metal oxides. Perchlorates can include perchlorates of alkali metals or alkaline earth metals, such as but not limited to, potassium perchlorate (KClO₄), potassium chlorate (KClO₃), lithium perchlorate (LiClO₄), sodium perchlorate (NaClO₄), and magnesium perchlorate (Mg(ClO₄)₂). In certain embodiments, transition metal oxides that function as metal-containing oxidizing agents include, but are not limited to, oxides of molybdenum, such as MoO₃; oxides of iron, such as Fe₂O₃; oxides of vanadium, such as V₂O₅; oxides of chromium, such as CrO₃ and Cr₂O₃; oxides of manganese, such as MnO₂; oxides of cobalt such as Co₃O₄; oxides of silver such as Ag₂O; oxides of copper, such as CuO; oxides of tungsten, such as WO₃; oxides of magnesium, such as MgO; and oxides of niobium, such as Nb₂O₅. In certain embodiments, the metal-containing oxidizing agent can include more than one metal-containing oxidizing agent.

In certain embodiments, a metal reducing agent and a metal-containing oxidizing agent can be in the form of a powder. The term “powder” refers to powders, particles, prills, flakes, and any other particulate that exhibits an appropriate size and/or surface area to sustain self-propagating ignition. For example, in certain embodiments, the powder can comprise particles exhibiting an average diameter ranging from 0.001 μm to 200 μm.

In certain embodiments, the amount of oxidizing agent in the initiator composition can be related to the molar amount of the oxidizer at or near the eutectic point for the fuel compositions. In certain embodiments, the oxidizing agent can be the major component and in others the metal reducing agent can be the major component. Also, as known in the art, the particle size of the metal and the metal-containing oxidizer can be varied to determine the burn rate, with smaller particle sizes selected for a faster burn (see, for example, PCT WO 2004/01396). Thus, in some embodiments where faster burn is desired, particles having nanometer scale diameters can be used.

In certain embodiments, the amount of metal reducing agent can range from 25% by weight to 75% by weight of the total dry weight of the initiator composition. In certain embodiments, the amount of metal-containing oxidizing agent can range from 25% by weight to 75% by weight of the total dry weight of the initiator composition.

In certain embodiments, an initiator composition can comprise at least one metal, such as those described herein, and at least one metal-containing oxidizing agent, such as, for example, a chlorate or perchlorate of an alkali metal or an alkaline earth metal, or metal oxide, and others disclosed herein.

In certain embodiments, an initiator composition can comprise at least one metal reducing agent selected from aluminum, zirconium, and boron. In certain embodiments, the initiator composition can comprise at least one oxidizing agent selected from molybdenum trioxide, copper oxide, tungsten trioxide, potassium chlorate, and potassium perchlorate.

In certain embodiments, aluminum can be used as a metal reducing agent. Aluminum can be obtained in various sizes such as nanoparticles, and can form a protective oxide layer and therefore can be commercially obtained in a dry state.

In certain embodiments, the initiator composition can include more than one metal reducing agent. In such compositions, at least one of the reducing agents can be boron. Examples of initiator compositions comprising boron are disclosed in U.S. Pat. Nos. 4,484,960, and 5,672,843. Boron can enhance the speed at which ignition occurs and thereby can increase the amount of heat produced by an initiator composition.

In certain embodiments, reliable, reproducible and controlled ignition of a fuel can be facilitated by the use of an initiator composition comprising a mixture of a metal containing oxidizing agent, at least one metal reducing agent and at least one binder and/or additive material such as a gelling agent and/or binder. The initiator composition can comprise the same or similar reactants at as those comprising a metal oxidation/reduction fuel, as disclosed herein.

In certain embodiments, an initiator composition can comprise one or more additive materials to facilitate, for example, processing, enhance the mechanical integrity and/or determine the burn and spark generating characteristics. An inert additive material will not react or will react to a minimal extent during ignition and burning of the initiator composition. This can be advantageous when the initiator composition is used in an enclosed system where minimizing pressure is useful. The additive materials can be inorganic materials and can function, for example, as binders, adhesives, gelling agents, thixotropic, and/or surfactants. Examples of gelling agents include, but are not limited to, clays such as LAPONITE, Montmorillonite, CLOISITE, metal alkoxides such as those represented by the formula R—Si(OR)ₙ and M(OR)ₙ where n can be 3 or 4, and M can be titanium, zirconium, aluminum, boron or other metal, and colloidal particles based on transition metal hydroxides or oxides. Examples of binding agents include, but are not limited to, soluble silicates such as sodium-silicates, potassium-silicates, aluminum silicates, metal alkoxides, inorganic polyanions, inorganic polycations, inorganic sol-gel materials such as alumina or silica-based sols. Other useful additive materials include glass beads, diatomaceous earth, nitrocellulose, polyvinylalcohol, guar gum, ethyl cellulose, cellulose acetate, polyvinylpyrrolidone, fluoro-carbon rubber (Viton) and other polymers that can function as a binder. In certain embodiments, the initiator composition can comprise more than one additive material.

In certain embodiments, additive materials can be useful in determining certain processing, ignition, and/or burn characteristics of an initiator composition. In certain embodiments, the particle size of the components of the initiator can be selected to tailor the ignition and burn rate characteristics as is known in the art, for example, as disclosed in U.S. Pat. No. 5,739,460.

In certain embodiments, it can be useful that the additives be inert. When sealed within an enclosure, the exothermic oxidation-reduction reaction of the initiator composition can generate an increase in pressure depending on the components selected. In certain applications, such as in portable medical devices, it can be useful to contain the pyrothermic materials and products of the exothermic reaction and other chemical reactions resulting from the high temperatures generated within the enclosure.

In certain embodiments particularly appropriate for use in medical applications, it is desirable that the additive not be an explosive, as classified by the U.S. Department of Transportation, such as, for example, nitrocellulose. In certain embodiments, the additives can be Viton or Laponite. These materials bind to the components of an initiator composition and can provide mechanical stability to the initiator composition.

The components of an initiator composition comprising the metal reducing agent, metal-containing oxidizing agent and/or additive materials and/or any appropriate aqueous- or organic-soluble binder, can be mixed by any appropriate physical or mechanical method to achieve a useful level of dispersion and/or homogeneity. For ease of handling, use and/or application, initiator compositions can be prepared as liquid suspensions or slurries in an organic or aqueous solvent.

The ratio of metal reducing agent to metal-containing oxidizing agent can be selected to determine the appropriate burn and spark generating characteristics. In certain embodiments, an initiator composition can be formulated to maximize the production of sparks having sufficient energy to ignite a fuel. Sparks ejected from an initiator composition can impinge upon the surface of a fuel, such as an oxidation/reduction fuel, causing the fuel to ignite in a self-sustaining exothermic oxidation-reduction reaction. In certain embodiments, the total amount of energy released by an initiator composition can range from 0.25 J to 8.5 J. In certain embodiments, a 20 μm to 100 μm thick solid film of an initiator composition can burn with a deflagration time ranging from 5 milliseconds to 30 milliseconds. In certain embodiments, a 40 μm to 100 μm thick solid film of an initiator composition can burn with a deflagration time ranging from 5 milliseconds to 20 milliseconds. In certain embodiments, a 40 μm to 80 μm thick solid film of an initiator composition can burn with a deflagration time ranging from 5 milliseconds to 10 milliseconds.

Examples of initiator compositions include compositions comprising 10% Zr, 22.5% B, 67.5% KClO₃; 49% Zr, 49% MoO₃, and 2% nitrocellulose; 33.9% Al, 55.4% MoO₃, 8.9% B, and 1.8% nitrocellulose; 26.5% Al, 51.5% MoO₃, 7.8% B, and 14.2% VITON; 47.6% Zr, 47.6% MoO₃, and 4.8% LAPONITE, where all percents are in weight percent of the total weight of the composition.

Examples of high-sparking and low gas producing initiator compositions comprise a mixture of aluminum, molybdenum trioxide, boron, and Viton. In certain embodiments, these components can be combined in a mixture of 20-30% aluminum, 40-55% molybdenum trioxide, 6-15% boron, and 5-20% Viton, where all percents are in weight percent of the total weight of the composition. In certain embodiments, an initiator composition comprises 26-27% aluminum, 51-52% molybdenum trioxide, 7-8% boron, and 14-15% Viton, where all percents are in weight percent of the total weight of the composition. In certain embodiments, the aluminum, boron, and molybdenum trioxide are in the form of nanoscale particles. In certain embodiments, the Viton is Viton A500.

In certain embodiments, the percussively activated initiator compositions can include compositions comprising a powdered metal-containing oxidizing agent and a powdered reducing agent comprising a central metal core, a metal oxide layer surrounding the core and a flurooalkysilane surface layer as disclosed, for example, in U.S. Pat. No. 6,666,936.

Typically, an initiator composition is prepared as a liquid suspension in an organic or aqueous solvent for coating the anvil and soluble binders are generally included to provide adhesion of the coating to the anvil.

A coating of an initiator composition can be applied to an anvil in various known ways. For example, an anvil can be dipped into a slurry of the initiator composition followed by drying in air or heat to remove the liquid and produce a solid adhered coating having the desired characteristic previously described. In certain embodiments, the slurry can be sprayed or spin coated on the anvil and thereafter processed to provide a solid coating. The thickness of the coating of the initiator composition on the anvil should be such, that when the anvil is placed in the enclosure, the initiator composition is a slight distance of around a few thousandths of an inch, for example, 0.004 inches, from the inside wall of the enclosure.

Percussive activation of an initiator composition can be effected by applying a forceful mechanical impact or blow against the side of an enclosure to deform the enclosure inwardly toward an anvil, to compress a coating of an initiator composition against the anvil. A mechanical impact sufficient to deform the tube can be provided by any appropriate mechanism.

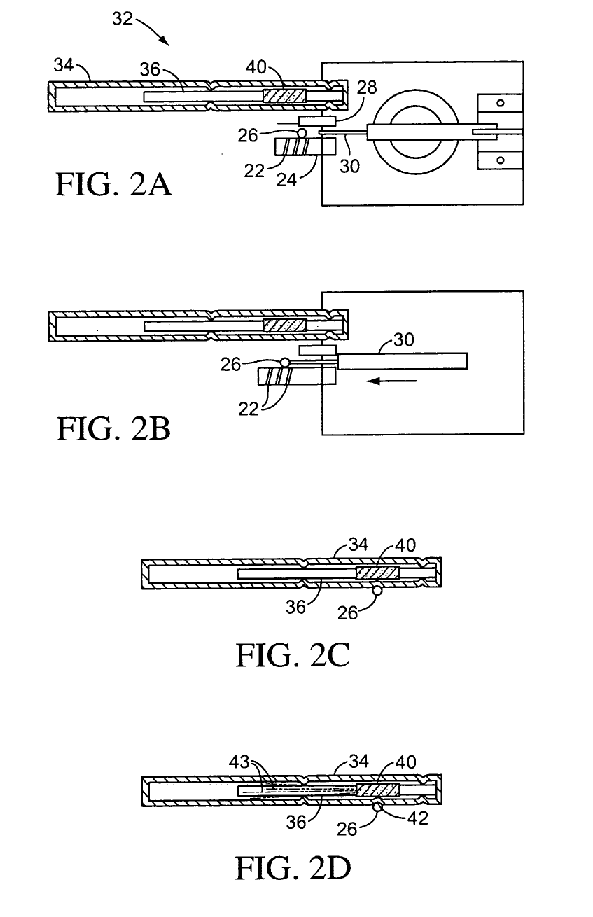

In certain embodiments, a mechanical impact can be provided by release of for example, but not limitation, a stressed torsion spring, compression spring, or a leaf spring. Such mechanisms are well known, for example, as mechanisms for percussively igniting photoflash lamps as disclosed, for example, in U.S. Pat. No. 4,146,356. For example, FIGS. 2A-2D shows a mechanism for actuating a percussively ignited system. Pre-stressed torsion spring 22 is mounted on torsion spring retainer 24, in proximity to a percussively activated igniter 32 (FIG. 2A). Percussively activated igniter 32 comprises a sealed enclosure 34, an anvil 36 disposed coaxially within enclosure 34 and held in place by indentations. An initiator composition 40 is disposed on a region of anvil 36. In a pre-release position, striker arm 26 of torsion spring 22 rests on a mechanical stop 28 (FIG. 2A). An engagement member 30 can be configured to push striker arm 26 off mechanical stop 28 to release striker arm 26 (FIGS. 2A & B). The stress in torsion spring 22 impels striker arm 26 to impact enclosure 34 adjacent initiator composition 40, which is disposed on anvil 36 (FIG. 2C). The impact force provided by striker arm 26 causes the wall of enclosure 34 to deform toward anvil 36 (FIG. 2D). The compression of initiator composition 40 between deformed enclosure wall 42 and anvil 36 causes initiator composition 40 to deflagrate and to eject sparks 43.

For use in inhalation devices, a mechanical impact mechanism, such as the stressed torsion spring illustrated in FIG. 2, can be coupled to an inhalation sensitive mechanism such that when a patient inhales on a medical device, the percussive ignition system will be activated. An inhalation sensitive mechanism includes mechanisms that are sensitive to pressure or air flow rate. An inhalation device can include a housing that defines an airway having at least one air inlet, and a mouthpiece having at least one air outlet. When a patient inhales on the mouthpiece, an air flow can be generated in the airway. The velocity of airflow within the airway can be sensed by an airflow velocity transducer such as a thermistor or mass flow sensor. Air flowing through the airway will also produce a difference in pressure between the outside and the inside of the airway. The pressure differential can be sensed by a pressure transducer such as a diaphragm.

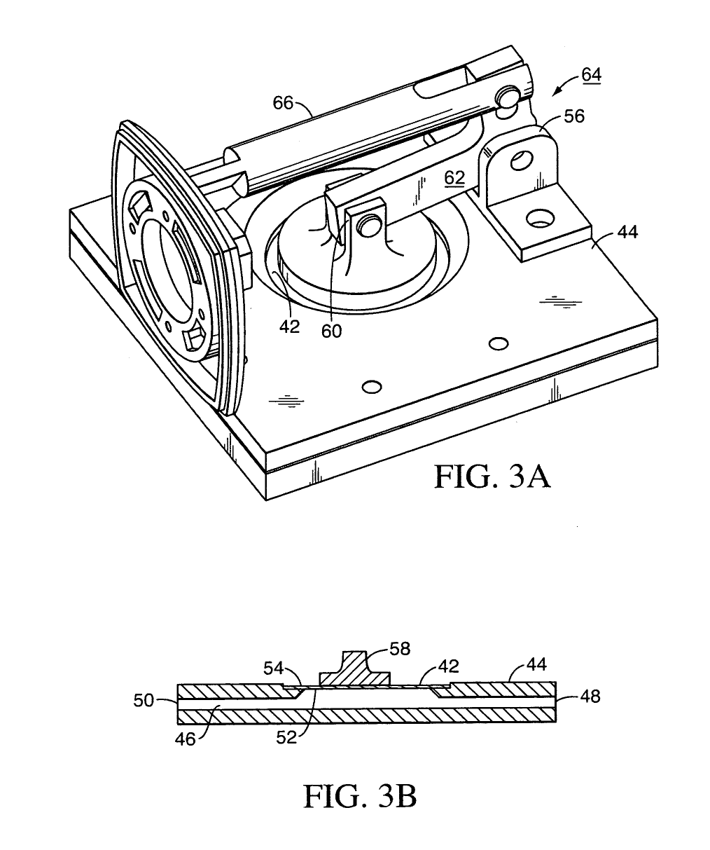

An airflow sensitive actuator for activating a percussive igniter is illustrated in FIGS. 3A-3B. FIGS. 3A shows an isometric view and FIG. 3B a cross-sectional view of an air flow sensitive actuator. FIG. 3 shows a diaphragm 42 incorporated into a housing 44. Housing 44 defines an airway 46 having an air inlet 48 and an air outlet 50. A first side 52 of diaphragm 42 is fluidly coupled to airway 46, and a second side 54 of diaphragm 42 is open to the ambient environment and mechanically coupled to lever arm assembly 56. Lever arm assembly 56 includes a mount 58 affixed to second side 54 of diaphragm 42, a pivot 60 attaching mount 58 to lever arm 62, and a fulcrum 64 connecting lever arm 62 to engagement arm 66. A flow of air through airway 46 can create a pressure differential across diaphragm 42. A pressure differential across diaphragm 42 caused by an air flow in airway 46 will result in diaphragm 42 being pulled toward airway 46. The motion of diaphragm 42, as translated through the mechanical lever and fulcrum assembly 56, will cause engagement arm 66 to move horizontally. Engagement arm 66 can return to its original position when air is no longer flowing through airway 46. The relative motion of engagement arm 66 can be used to release a pre-stressed torsion spring, for example, as illustrated in FIGS. 2A-2D. For example, as illustrated in FIGS. 2A-2D, the relative motion of engagement arm 66 can push striker arm 26 off mechanical stop 28 and thereby release striker arm 26 to impact enclosure 34. In certain embodiments, the motion of the engagement arm itself can provide a mechanical impact sufficient to percussively activate an initiator composition. As will be appreciated by those skilled in the art, other mechanical mechanisms can be used to provide relative motion of an engagement arm upon deflection of a diaphragm.

Diaphragm 42 can be a flexible membrane fabricated from any appropriate material. For example, diaphragm 42 can be a thin elastomeric membrane having a thickness ranging from 0.001 inches to 0.1 inches. Examples of suitable diaphragm materials include nitrile rubber, silicon rubber, thin metals, and the like. The mechanical force produced by the diaphragm will at least in part be determined by the area of the section of the diaphragm fluidly coupled to the airway, and air flow velocity in the airway which produces a proportional pressure differential across the membrane. For example, a diaphragm having a surface area of 1.75 in² with a 2:1 lever ratio at a pressure drop of 10 cm H₂O will generate a force of about 220 grams. This force will vary, however, depending on for example the orifice size and geometry.

The inhalation actuated percussive ignition system can be used to ignite a fuel, such as a fuel comprising a metal reducing agent and a metal-containing oxidizing agent. A metal oxidation-reduction fuel and percussive ignition system can be incorporated into a compact, manufacturable, heat package.

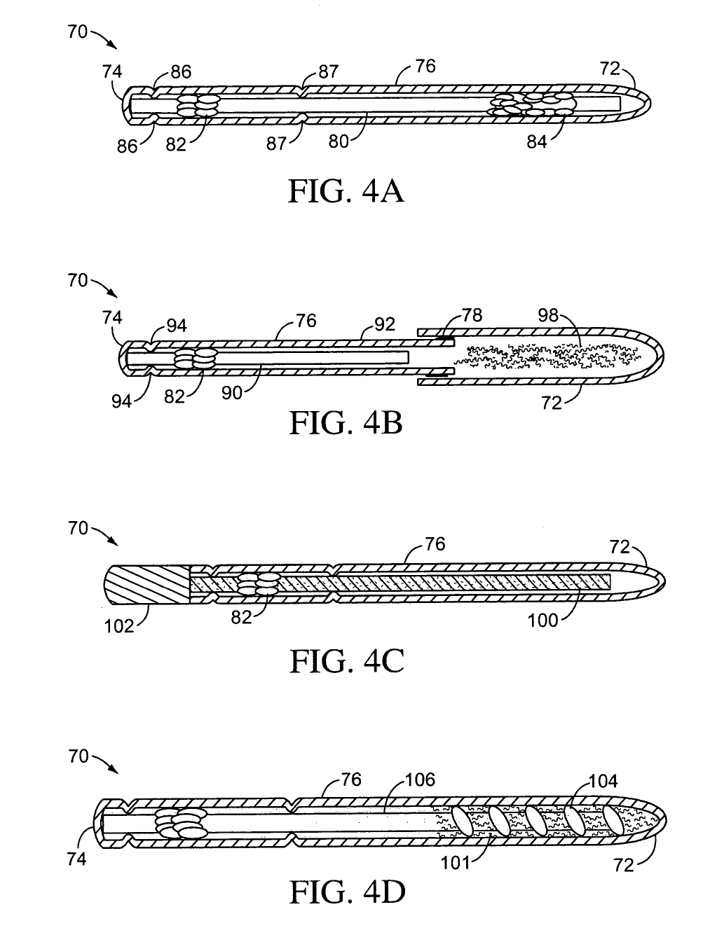

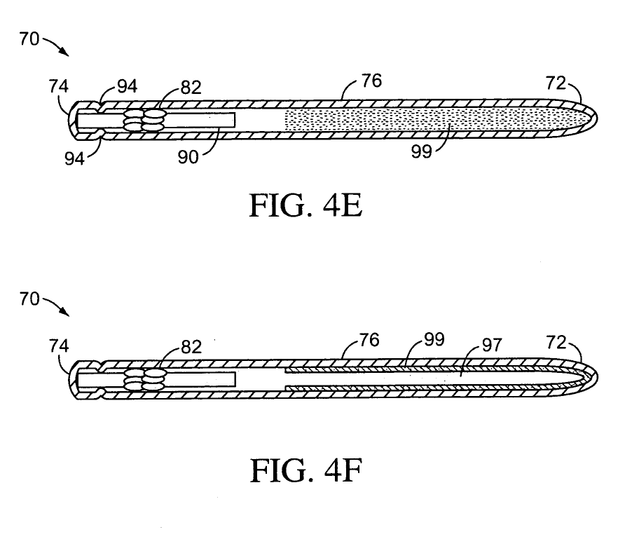

FIGS. 4A-4F show embodiments of heat packages comprising a percussive igniter. The heat packages 70 shown in FIGS. 4A-4F substantially comprise a sealed tube or cylinder 76 having a first end 72 and a second end 74. For use in a portable medical device, it is important that a heat package remain sealed when ignited and withstand any internal pressure generated by the burning fuels. In FIGS. 4A, and 4C-4F, first end 72 of heat package 70 is integral with the tubular body portion 76 or formed from the same part as tubular body portion 76. In FIG. 4B, first end 72 is a separate section and second end 74 is a separate section. Sections 72, 74 can be sealed at interface 78 by any appropriate means capable of withstanding the pressure and temperatures generated during combustion of the initiator and fuel compositions such as by soldering, welding, crimping, adhesively affixing, mechanically coupling, or the like. Second end 74 can also be sealed by similar means, and in certain embodiments, can include an insert, which may be thermally conductive or non-conductive.

FIG. 4A shows an embodiment of a heat package 70 having a coaxially positioned anvil 80 held in place by indentations 86, 87. Anvil 80 extends substantially the length of heat package 70. A thin coating of an initiator composition 82 is disposed toward one end of anvil 80, and a coating of a metal oxidation/reduction fuel composition 84 as disclosed herein is disposed on the other end of anvil 80. Indentations 87 provide space between anvil 80 and the inner wall of tube 70 to allow sparks produced during deflagration of initiator composition 82 to strike and ignite fuel composition 84. Anvil 80 can include features to facilitate retention of a greater amount of fuel and/or to facilitate assembly. For example, the end of anvil 80 on which fuel 84 is disposed can include fins or serrations to increase the surface area.

FIG. 4B shows an embodiment of a heat package 70 having an anvil 90 extending less than the length of heat package 70. Anvil 90 is held coaxially within tube 92 by indentations 94 toward one end of anvil 90. Minimizing or eliminating obstructions in the space between anvil 90 and the inner wall of tube 92 can facilitate the ability of sparks ejected from initiator composition 82 to strike and ignite fuel 98. First and second sections 72, 74 forming heat package 70 shown in FIG. 4B are sealed at interface 78. A fuel 98 is disposed within first section 72. Short anvil 90 permits the entire area within first section 72 to be filled with fuel 98.

In FIG. 4C, anvil 100 comprises a fuel. Initiator composition 82 is disposed on part of the surface of anvil 100. Activation of initiator composition 82 can cause anvil 100 to ignite. End section 102 can be made of a thermally insulating material to facilitate mounting heat package 70. Use of a fuel extending substantially the length of the heat package can provide a larger usefully heated area.

FIG. 4D shows an embodiment of heat package 70 in which the front end 104 of anvil 106 is formed with a high-pitch, thin-wall auger which can be used, for example, to load fuel into cylinder end 72. Such a design can be useful in facilitating manufacturability of the heat package.

FIG. 4E shows an embodiment of heat package 70 in which anvil 90 extends part of the length of tube 76, and a substantial part of the interior of tube 76 is filled with a fuel 99. Filing a substantial part of tube 76 with fuel 99 can increase the amount of heat generated by heat package 70. As shown in FIG. 4F, in certain embodiments, fuel 99 can be disposed as a layer on the inside wall of tube 76 and the center region 97 can be a space. A layer of fuel 99 can facilitate even heating of tube 76 and/or more rapidly reaching a maximum temperature by exposing a larger surface area that can be ignited by sparks ejected from initiator composition 82. A space in center region 97 can provide a volume in which released gases can accumulate to reduce the internal pressure of heat package 70.

Heat packages, such as shown in FIGS. 4A-F can have any appropriate dimension which can at least in part determined by the surface area intended to be heated and the maximum desired temperature. Percussively activated heat packages can be particularly useful as compact heating elements capable of generating brief heat impulses such as can be used to vaporize a drug to produce a condensation aerosol for inhalation. In such applications, the length of a heat package can range from 0.4 inches to 2 inches and have a diameter ranging from 0.3 inches to 0.1 inches. The optimal dimensions of the anvil, the dimensions of the enclosed cylinder, and the amount of fuel disposed therein for a particular application and/or use can be determined by standard optimization procedures.

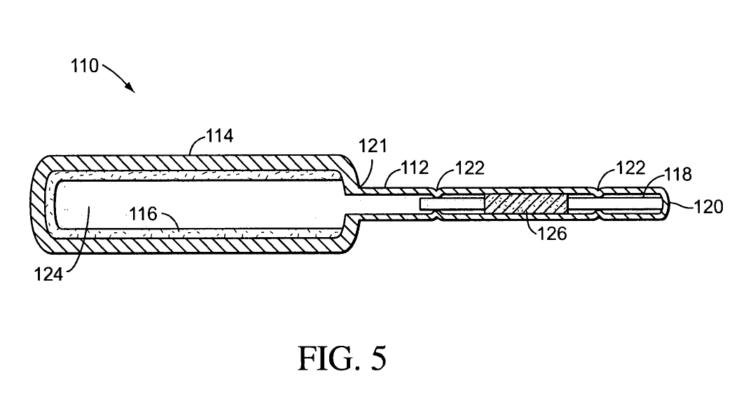

FIG. 5 shows another embodiment of a heat package. Heat package 110 includes a first section 112 comprising a percussive ignition system, and a second section 114 having a cross-sectional dimension greater than that of first section 112 comprising a fuel 116. The percussive ignition system includes anvil 118 coaxially disposed within a deformable tube 112. One end 120 of deformable tube is sealed and the opposing end 121 is joined to section 114. Anvil 118 is held in place by indentations 122. A part of anvil 118 is coated with an initiator composition 126. Second section 114 comprises an enclosure having a wall thickness and cross-sectional dimension greater than that of first section 112. Such a design may be useful to increase the amount of fuel, to increase the external surface area on which a substance can be disposed, to provide a volume in which gases can expand to reduce the pressure within the enclosure, to provide a greater fuel surface area for increasing the burn rate, and/or to increase the structural integrity of the first section. In FIG. 5, fuel 116 is shown as a thin layer disposed along the inner wall of second section 114. Other fuel configurations are possible. For example, the fuel can be disposed only along the horizontal walls, can completely or partially fill internal area 124, and/or be disposed within a fibrous matrix disposed throughout area 124. It will be appreciated that the shape, structure and composition of fuel 116 can be determined as appropriate for a particular application that, in part, will be determined by the thermal profile desired.

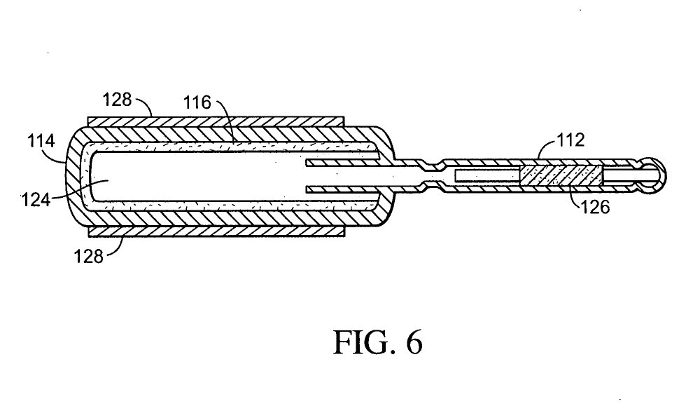

FIG. 6 shows a further embodiment of a heat package. The heat package illustrated in FIG. 6 is similar to that shown in FIG. 5 with the principle difference that deformable tube 112 extends into area 124 of second section 114. The configuration illustrated in FIG. 6 can be useful for enhancing and/or controlling the distribution of sparks generated by deflagration of initiator composition 126. The heat package illustrated in FIG. 6 also shows a substance 128 disposed on the outer surface of second section 114. As disclosed herein, percussively activated initiator composition 126 can ignite fuel 116. The heat generated by the burning of fuel 116 can be transferred to second section 114 can vaporize substance 128.

The fuel can comprise a metal reducing agent an oxidizing agent, such as, for example, a metal-containing oxidizing agent.

In certain embodiments, the fuel can comprise a mixture of Zr and MoO₃, Zr and Fe2O₃, Al and MoO₃, or Al and Fe₂O₃. In certain embodiments, the amount of metal reduction agent can range form 60% by with to 90% by weight, and the amount of metal containing oxidizing agent can range from 40% by weight to 10% by weight.

Examples of useful metal reducing agents for forming a fuel include, but are not limited to, molybdenum, magnesium, calcium, strontium, barium, boron, titanium, zirconium, vanadium, niobium, tantalum, chromium, tungsten, manganese, iron, cobalt, nickel, copper, zinc, cadmium, tin, antimony, bismuth, aluminum, and silicon. In certain embodiments, a metal reducing agent can be selected from aluminum, zirconium, and titanium. In certain embodiments, a metal reducing agent can comprise more than one metal reducing agent.

In certain embodiments, an oxidizing agent for forming a fuel can comprise oxygen, an oxygen based gas, and/or a solid oxidizing agent. In certain embodiments, an oxidizing agent can comprise a metal-containing oxidizing agent. In certain embodiments, a metal-containing oxidizing agent includes, but is not limited to, perchlorates and transition metal oxides. Perchlorates can include perchlorates of alkali metals or alkaline earth metals, such as but not limited to, potassium perchlorate (KClO₄), potassium chlorate (KClO₃), lithium perchlorate (LiClO₄), sodium perchlorate (NaClO₄), and magnesium perchlorate (Mg(ClO₄)₂). In certain embodiments, transition metal oxides that function as oxidizing agents include, but are not limited to, oxides of molybdenum, such as MoO₃; iron, such as Fe₂O₃; vanadium, such as V₂O₅; chromium, such as CrO₃ and Cr₂O₃; manganese, such as MnO₂; cobalt such as Co₃O₄; silver such as Ag₂O; copper, such as CuO; tungsten, such as WO₃; magnesium, such as MgO; and niobium, such as Nb₂O₅. In certain embodiments, the metal-containing oxidizing agent can include more than one metal-containing oxidizing agent.

In certain embodiments, the metal reducing agent forming the solid fuel can be selected from zirconium and aluminum, and the metal-containing oxidizing agent can be selected from MoO₃ and Fe₂O₃.

The ratio of metal reducing agent to metal-containing oxidizing agent can be selected to determine the ignition temperature and the burn characteristics of the solid fuel. An exemplary chemical fuel can comprise 75% zirconium and 25% MoO₃, percentage by weight. In certain embodiments, the amount of metal reducing agent can range from 60% by weight to 90% by weight of the total dry weight of the solid fuel. In certain embodiments, the amount of metal-containing oxidizing agent can range from 10% by weight to 40% by weight of the total dry weight of the solid fuel.

In certain embodiments, a fuel can comprise one or more additive materials to facilitate, for example, processing and/or to determine the thermal and temporal characteristics of a heating unit during and following ignition of the fuel. An additive material can be inorganic materials and can function as binders, adhesives, gelling agents, thixotropic, and/or surfactants. Examples of gelling agents include, but are not limited to, clays such as Laponite, Montmorillonite, Cloisite, metal alkoxides such as those represented by the formula R—Si(OR)ₙ and M(OR)ₙ where n can be 3 or 4, and M can be titanium, zirconium, aluminum, boron or other metal, and colloidal particles based on transition metal hydroxides or oxides. Examples of binding agents include, but are not limited to, soluble silicates such as sodium-silicates, potassium-silicates, aluminum silicates, metal alkoxides, inorganic polyanions, inorganic polycations, inorganic sol-gel materials such as alumina or silica-based sols. Other useful additive materials include glass beads, diatomaceous earth, nitrocellulose, polyvinylalcohol, guar gum, ethyl cellulose, cellulose acetate, polyvinylpyrrolidone, fluorocarbon rubber (VITON) and other polymers that can function as a binder.

Other useful additive materials include glass beads, diatomaceous earth, nitrocellulose, polyvinylalcohol, and other polymers that may function as binders. In certain embodiments, the fuel can comprise more than one additive material. The components of the fuel comprising the metal, oxidizing agent and/or additive material and/or any appropriate aqueous- or organic-soluble binder, can be mixed by any appropriate physical or mechanical method to achieve a useful level of dispersion and/or homogeneity. In certain embodiments, the fuel can be degassed.

The fuel in the heating unit can be any appropriate shape and have any appropriate dimensions. The fuel can be prepared as a solid form, such as a cylinder, pellet or a tube, which can be inserted into the heat package. The fuel can be deposited into the heat package as a slurry or suspension which is subsequently dried to remove the solvent. The fuel slurry or suspension can be spun while being dried to deposit the fuel on the inner surface of the heat package. In certain embodiments, the fuel can be coated on a support, such as the anvil by an appropriate method, including, for example, those disclosed herein for coating an initiator composition on an anvil.

In certain embodiments the anvil can be formed from a combustible metal alloy or metal/metal oxide composition, such as are known in the art, for example, Pyrofuze (available from Sigmund Cohn). Examples of fuel compositions suitable for forming the anvil are disclosed in U.S. Pat. Nos, 3,503,814; 3,377,955; and PCT Application No. WO 93/14044, the pertinent parts of each of which are incorporated herein by reference. In embodiments, when the anvil is formed form a combustible material, no additional fuel other than an initiator is needed.

In certain embodiments, the fuel can be supported by a malleable fibrous matrix which can be packed into the heat package. The fuel comprising a metal reducing agent and a metal-containing oxidizing agent can be mixed with a fibrous material to form a malleable fibrous fuel matrix. A fibrous fuel matrix is a convenient fuel form that can facilitate manufacturing and provides faster burn rates. A fibrous fuel matrix is a paper-like composition comprising a metal oxidizer and a metal-containing reducing agent in powder form supported by an inorganic fiber matrix. The inorganic fiber matrix can be formed from inorganic fibers, such as ceramic fibers and/or glass fibers. To form a fibrous fuel, the metal reducing agent, metal-containing oxidizing agent, and inorganic fibrous material are mixed together in a solvent, and formed into a shape or sheet using, for example, paper-making equipment, and dried. The fibrous fuel can be formed into mats or other shapes as can facilitate manufacturing and/or burning.

The heat packages can have any appropriate dimensions. The self-contained heat packages are particularly suited for applications where small size and safety are useful, such as in medical device applications. In certain embodiments, the length of a heat package can range from 0.8 inches to 2 inches, and the width of the heat package can range from 0.02 inches to 0.2 inches. In certain embodiments, the width of the anvil can range from 0.005 inches to 0.19 inches.

The self-contained heat packages can be percussively ignited by mechanically impacting the enclosure with sufficient force to cause the part of the enclosure to be directed toward the anvil, wherein the initiator composition is compressed between the tube and the anvil. The compressive force initiates deflagration of the initiator composition. Sparks produced by the deflagration are directed toward and impact the fuel composition, causing the fuel composition to ignite in a self-sustaining metal oxidation reaction generating a rapid, intense heat impulse.

In certain embodiments, a substance can be disposed on the outer surface of the percussively activated heat package. When activated, the heat generated by burning of the fuel can provide a rapid, intense thermal impulse capable of vaporizing a solid thin film of substance disposed on an exterior surface of the heat package with minimal degradation. A solid thin film of a substance can be applied to the exterior of a heat package by any appropriate method and can depend in part on the physical properties of the substance and the final thickness of the layer to be applied. In certain embodiments, methods of applying a substance to a heat package include, but are not limited to, brushing, dip coating, spray coating, screen printing, roller coating, inkjet printing, vapor-phase deposition, spin coating, and the like. In certain embodiments, the substance can be prepared as a solution comprising at least one solvent and applied to an exterior surface of a heat package. In certain embodiments, a solvent can comprise a volatile solvent such as acetone, or isopropanol. In certain embodiments, the substance can be applied to a heat package as a melt. In certain embodiments, a substance can be applied to a film having a release coating and transferred to a heat package. For substances that are liquid at room temperature, thickening agents can be admixed with the substance to produce a viscous composition comprising the substance that can be applied to a support by any appropriate method, including those described herein. In certain embodiments, a layer of substance can be formed during a single application or can be formed during repeated applications to increase the final thickness of the layer.

In certain embodiments, a substance disposed on a heat package can comprise a therapeutically effective amount of at least one physiologically active compound or drug. A therapeutically effective amount refers to an amount sufficient to effect treatment when administered to a patient or user in need of treatment. Treating or treatment of any disease, condition, or disorder refers to arresting or ameliorating a disease, condition or disorder, reducing the risk of acquiring a disease, condition or disorder, reducing the development of a disease, condition or disorder or at least one of the clinical symptoms of the disease, condition or disorder, or reducing the risk of developing a disease, condition or disorder or at least one of the clinical symptoms of a disease or disorder. Treating or treatment also refers to inhibiting the disease, condition or disorder, either physically, e.g. stabilization of a discernible symptom, physiologically, e.g., stabilization of a physical parameter, or both, and inhibiting at least one physical parameter that may not be discernible to the patient. Further, treating or treatment refers to delaying the onset of the disease, condition or disorder or at least symptoms thereof in a patient which may be exposed to or predisposed to a disease, condition or disorder even though that patient does not yet experience or display symptoms of the disease, condition or disorder.

In certain embodiments, the amount of substance disposed on a support can be less than 100 micrograms, in certain embodiments, less than 250 micrograms, and in certain embodiments, less than 1,000 micrograms. In certain embodiments, the thickness of a solid thin film applied to a heat package can range from 0.01 μm to 20 μm, and in certain embodiments can range from 0.5 μm to 10 μm.

In certain embodiments, a substance can comprise a pharmaceutical compound. In certain embodiments, the substance can comprise a therapeutic compound or a non-therapeutic compound. A non-therapeutic compound refers to a compound that can be used for recreational, experimental, or pre-clinical purposes. Classes of drugs that can be used include, but are not limited to, anesthetics, anticonvulsants, antidepressants, antidiabetic agents, antidotes, antiemetics, antihistamines, anti-infective agents, antineoplastics, antiparkinsonian drugs, antirheumatic agents, antipsychotics, anxiolytics, appetite stimulants and suppressants, blood modifiers, cardiovascular agents, central nervous system stimulants, drugs for Alzheimer's disease management, drugs for cystic fibrosis management, diagnostics, dietary supplements, drugs for erectile dysfunction, gastrointestinal agents, hormones, drugs for the treatment of alcoholism, drugs for the treatment of addiction, immunosuppressives, mast cell stabilizers, migraine preparations, motion sickness products, drugs for multiple sclerosis management, muscle relaxants, nonsteroidal anti-inflammatories, opioids, other analgesics and stimulants, ophthalmic preparations, osteoporosis preparations, prostaglandins, respiratory agents, sedatives and hypnotics, skin and mucous membrane agents, smoking cessation aids, Tourette's syndrome agents, urinary tract agents, and vertigo agents.

While it will be recognized that extent and dynamics of thermal degradation can at least in part depend on a particular compound, in certain embodiments, thermal degradation can be minimized by rapidly heating the substance to a temperature sufficient to vaporize and/or sublime the active substance. In certain embodiments, the substrate can be heated to a temperature of at least 250° C. in less than 500 msec, in certain embodiments, to a temperature of at least 250° C. in less than 100 msec, and in certain embodiments, to a temperature of at least 250° C. in less than 250 msec.

In certain embodiments, rapid vaporization of a layer of substance can occur with minimal thermal decomposition of the substance, to produce a condensation aerosol exhibiting high purity of the substance. For example, in certain embodiments, less than 10% of the substance is decomposed during thermal vaporization, and in certain embodiments, less than 5% of the substance is decomposed during thermal vaporization.

Examples of drugs that can be vaporized from a heated surface to form a high purity aerosol include aluterol, alprazolam, apomorphine HCl, aripiprazole, atropine, azatadine, benztropine, bromazepam, brompheniramine, budesonide, bumetanide, buprenorphine, butorphanol, carbinoxamine, chloridiazepoxide, chlorpheniramine, ciclesonide, clemastine, clonidine, colchicine, cyproheptadine, diazepam, donepezil, eletriptan, estazolam, estradiol, fentanyl, flumazenil, flunisolide, flunitrazepam, fluphenazine, fluticasone propionate, frovatriptan, galanthamine, granisetron, hydromorphone, hyoscyamine, ibutilide, ketotifen, loperamide, melatonin, metaproterenol, methadone, midazolam, naratriptan, nicotine, oxybutynin, oxycodone, oxymorphone, pergolide, perphenazine, pindolol, pramipexole, prochlorperazine, rizatriptan, ropinirole, scopolamine, selegiline, tadalafil, terbutaline, testosterone, tetrahydrocannabinol, tolterodine, triamcinolone acetonide, triazolam, trifluoperazine, tropisetron, zaleplon, zolmitriptan, and zolpidem. These drugs can be vaporized from a thin film having a thickness ranging from 0.2 μm to 7 μm, and corresponding to a coated mass ranging from 0.2 mg to 40 mg, upon heating the thin film of drug to a temperature ranging from 250° C. to 550° C. within less than 100 msec, to produce aerosols having a drug purity greater than 90% and in many cases, greater than 99%.

### EXAMPLES

Embodiments of the present disclosure can be further defined by reference to the following examples, which describe in detail preparation of the compounds of the present disclosure. It will be apparent to those skilled in the art that many modifications, both to the materials and methods, may be practiced without departing from the scope of the present disclosure.

### Example 1

### Percussive Ignition Using Initiator Composition

The preparation of a heating unit according to FIG. 8 using percussive ignition is described.

To prepare the percussive ignition system, a one-quarter section of a thin stainless steel wire anvil was dip coated in an initiator composition of 26.5% Al, 51.4% MoO₃, 7.7% B, and 14.3% Viton A500 weight percent based on dry weight, in amyl acetate. The coated wire was then dried at 40° C. to 50° C. for 1 hour. The dried, coated wire anvil was placed into a 0.003 inch thick aluminum ignition tube, and one end of the tube was crimped to hold the wire substantially coaxial within the tube.

In another embodiment, the initiator composition was formed by combining 620 parts by weight of titanium having a particle size less than 20 um, 100 parts by weight of potassium chlorate, 180 parts by weight red phosphorous, 100 parts by weight sodium chlorate, and 620 parts by weight water, and 2% polyvinyl alcohol binder.

### Example 2

### Percussively Ignited Heat Package

The ignition assembly comprising a ¼ inch section of a thin stainless steel wire anvil was dip coated with the initiator composition and dried at about 40-50 C for about 1 hour. The dried, coated wire anvil was inserted into a 0.003 inch thick, soft walled aluminum tube. The tube was crimped to hold the wire anvil in place.

Other embodiments of the present disclosure will be apparent tot those skilled in the art from consideration of the specification and practice of the present disclosure disclosed herein. It is intended that the specification and examples be considered as exemplary only, with a true scope and spirit of the present disclosure being indicated by the following claims.

What is claimed is:

1. An inhalation actuated percussive ignition system comprising:
a housing defining an airway, wherein the housing comprises at least one air inlet and a mouthpiece having at least one air outlet;
an airflow sensitive actuator coupled to the airway; and
a mechanism coupled to the airflow sensitive actuator configured to activate a percussive igniter;
wherein the percussive igniter is activated by an airflow in the airway produced by inhaling through the mouthpiece.

2. The ignition system of claim 1, wherein the airway supports an air flow rate ranging from about 10 L/min to about 200 L/min.

3. The ignition system of claim 1, wherein the airflow sensitive actuator is activated by pressure or airflow rate.

4. The ignition system of claim 1, wherein the airflow sensitive actuator is activated by an airflow rate.

5. The ignition system of claim 1, wherein the airflow sensitive actuator is activated by a pressure differential.

6. The ignition system of claim 5, wherein the airflow sensitive actuator comprises a diaphragm.

7. The ignition system of claim 6, wherein the area of the diaphragm and the pressure differential across the diaphragm caused by an airflow produces a mechanical force sufficient to activate the percussive igniter.

8. The ignition system of claim 1, wherein the mechanism configured to activate the percussive igniter produces a mechanical impact.

9. The ignition system of claim 1, wherein the mechanism configured to activate the percussive igniter comprises a spring, a mechanism for stressing the spring, and a mechanism for releasing the spring to mechanically impact the percussive igniter.

10. The ignition system of claim 1, wherein the percussive igniter comprises:
a deformable part;
an anvil disposed adjacent the deformable part; and
an initiator composition disposed between the anvil and the deformable part;
wherein the initiator composition is ignited when an impact on the deformable part compresses the initiator composition against the anvil.

11. The ignition system of claim 10, wherein the initiator composition comprises at least one metal reducing agent, a metal-containing oxidizing agent, and at least one binder.

12. The ignition system of claim 1, wherein the airflow sensitive actuator activates the percussive igniter when the airflow rate is at least about 10 L/min.

13. A method for activating a percussive igniter, comprising the steps of:
providing an inhalation actuated percussive ignition system, comprising:
a housing defining an airway, wherein the housing comprises at least one air inlet and a mouthpiece having at least one air outlet;
an airflow sensitive actuator coupled to the airway; and
a mechanism coupled to the airflow sensitive actuator configured to activate a percussive igniter;
inhaling through the mouthpiece to generate an air flow in the airway;
actuating the airflow sensitive actuator; and
activating the percussive igniter.

14. An inhalation actuated percussive ignition system comprising:
a housing defining an airway, wherein the housing comprises at least one air inlet and a mouthpiece having at least one air outlet;
an airflow sensitive actuator coupled to the airway; and
a mechanism coupled to the airflow sensitive actuator configured to activate a percussive heating element;
wherein the percussive heating element comprises an enclosure comprising a region capable of being deformed by a mechanical impact;
an anvil disposed within the enclosure;
a percussive initiator composition disposed within the enclosure, wherein the initiator composition is configured to be ignited when the deformable region of the enclosure is deformed; and
a fuel disposed within the enclosure configured to be ignited by the initiator composition; and
wherein the percussive heating element is activated by an airflow in the airway produced by inhaling through the mouthpiece.

15. The heating element of claim 14, wherein a part of the external surface of the enclosure reaches a temperature of at least 200° C. in less than 200 msec following activation of the percussive igniter.

16. The heating element of claim 14, wherein the enclosure remains sealed following burning of the fuel.

17. The heating element of claim 14, wherein the enclosure comprises a sealed tube.

18. The heating element of claim 14, wherein the enclosure comprises a metal.

19. The heating element of claim 14, wherein the deformable region of the enclosure is deformable at a force ranging from about 0.5 in-lb to about 3.0 in-lb.

20. The heating element of claim 14, wherein the anvil comprises a solid rod, pin or wire.

21. The heating element of claim 14, wherein the anvil is coaxially disposed in the center of the enclosure.

22. The heating element of claim 14, wherein the anvil comprises the fuel.

23. The heating element of claim 14, wherein the initiator composition comprises a metal-containing oxidizing agent, at least one metal reducing agent, and a non-explosive binder.

24. The heating element of claim 14, wherein the initiator composition is disposed between the inner wall of the enclosure and the anvil.

25. The heating element of claim 14, wherein the initiator composition is disposed on the surface of the anvil.

26. The heating element of claim 14, wherein the initiator composition does not contact the inner wall of the enclosure until the deformable region is deformed.

27. The heating element of claim 14, wherein the initiator composition is disposed on the surface of the anvil adjacent a deformable region of the enclosure.

28. The heating element of claim 14, wherein the fuel comprises at least one metal reducing agent and at least one metal-containing oxidizing agent.

29. The heating element of claim 28, wherein the fuel further comprises at least one inert material.

30. The heating element of claim 14, wherein the fuel comprises a mixture of a metal reducing agent, a metal-containing oxidizing agent, and an inert fibrous material.

31. The heating element of claim 30, wherein the inert fibrous material is glass fiber.

32. The heating element of claim 14, wherein the length of the enclosure ranges from about 0.8 inches to about 2 inches.

33. The heating element of claim 14, wherein the width of the enclosure ranges from about 0.02 inches to about 0.2 inches.

34. The heating element of claim 14, wherein the width of the anvil ranges from about 0.005 inches to about 0.19 inches.

35. The heating element of claim 14, wherein a solid thin film comprising a drug is disposed on at least a portion of the exterior surface of the enclosure.

36. The heating element of claim 35, wherein the drug is selected from at least one of the following: aluterol, alprazolam, apomorphine HCl, aripiprazole, atropine, azatadine, benztropine,bromazepam, brompheniramine, budesonide, bumetanide, buprenorphine, butorphanol, carbinoxamine, chloridiazepoxide, chlorpheniramine, ciclesonide, clemastine, clonidine, colchicine, cyproheptadine, diazepam, donepezil, eletriptan, estazolam, estradiol, fentanyl, flumazenil, flunisolide, flunitrazepam, fluphenazine, fluticasone propionate, frovatriptan, galanthamine, granisetron, hydromorphone, hyoscyamine, ibutilide, ketotifen, loperamide, melatonin, metaproterenol, methadone, midazolam, naratriptan, nicotine, oxybutynin, oxycodone, oxymorphone, pergolide, perphenazine, pindolol, pramipexole, prochlorperazine, rizatriptan, ropinirole, scopolamine, selegiline, tadalafil, terbutaline, testosterone, tetrahydrocannabinol, tolterodine, triamcinolone acetonide, triazolam, trifluoperazine, tropisetron, zaleplon, zolmitriptan, and zolpidem.

37. The heating element of claim 35, wherein the thickness of the solid thin film ranges from about 0.1 μm to about 20 μm.

38. An inhalation actuated heating system, comprising:
a housing defining an airway, wherein the housing comprises at least one air inlet and a mouthpiece having at least one air outlet;
an airflow sensitive actuator coupled to the airway;
a mechanism coupled to the airflow sensitive actuator configured to activate a percussive igniter; and
a heating element comprising a fuel, wherein the fuel is configured to be ignited by the percussive igniter;
wherein the percussive igniter is activated by an airflow in the airway produced by inhaling through the mouthpiece.

39. The heating system of claim 38, wherein the heating element is disposed within the airway.

40. The heating element of claim 38, wherein the airflow sensitive actuator comprises a diaphragm.

41. The heating element of claim 38, wherein the area of the diaphragm and a pressure differential across the diaphragm caused by the airflow produces a mechanical force sufficient to activate the percussive igniter.

42. The heating element of claim 38, wherein the mechanism configured to activate the percussive igniter produces a mechanical impact.

43. The heating element of claim 33, wherein the mechanism configured to activate the percussive igniter comprises a spring, a mechanism for stressing the spring, and a mechanism for releasing the spring to mechanically impact the percussive igniter.

44. A method of actuating a heating element, comprising:
inhaling to generate an airflow;
actuating an airflow sensitive actuator disposed within the air flow;
activating a percussive igniter coupled to the air flow sensitive actuator; and
igniting a fuel.

45. A method of producing a condensation aerosol of a substance, comprising:
providing an inhalation actuated heating element comprising:
a housing defining an airway, wherein the housing comprises at least one air inlet and a mouthpiece having at least one air outlet;
an airflow sensitive actuator coupled to the airway;
a mechanism coupled to the airflow sensitive actuator configured to activate a percussive igniter;
a heating element disposed within the airway, wherein the heating element comprises a fuel disposed within the enclosure, and a percussive igniter disposed within the enclosure and configured to ignite the fuel; and
a substance disposed on at least a portion of the exterior of the enclosure;
inhaling through the mouthpiece to generate an airflow in the airway;
actuating the airflow sensitive actuator;
activating the percussive igniter;
igniting the fuel; and
vaporizing the substance disposed on exterior of the enclosure to form an aerosol comprising the substance in the airway.
